(19) United States
(12) Patent Application Publication
(10) Pub. No.: US 2006/0035982 A1
(43) Pub. Date: Feb. 16, 2006

(54) Method for promoting blood stream

(75) Inventors: Jonathan Warr, Paris (FR); Satomi Kunieda, Hiratsuka-shi (JP)

Correspondence Address: SUGHRUE MION, PLLC, 2100 PENNSYLVANIA AVENUE, N.W., SUITE 800, WASHINGTON, DC, 20037, US

(73) Assignee: TAKASAGO INTERNATIONAL CORPORATION

(21) Appl. No.: 11/198,348

(22) Filed: Aug. 8, 2005

(30) Foreign Application Priority Data
Aug. 6, 2004 (FR) 04/08725

(51) Int. Cl. A61K 31/05; A61K 31/075

(52) U.S. Cl. 514/718; 514/734

(57) ABSTRACT

The present invention has as its object a method for topically promoting blood stream of a subject, which comprises applying at least one compound of formula (A)
wherein 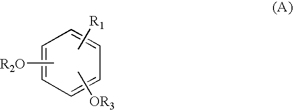
R₁ represents a hydrogen, a hydroxy group, a C₁-C₄ alkyl group or a C₁-C₃ alkoxy group, and
R₂ and R₃, each independently represent a hydrogen or a C₁-C₄ alkyl group, to the subject.

### FIELD OF THE INVENTION

The present invention relates to a method for promoting blood stream.

### BACKGROUND OF THE INVENTION

It has been known for a long time that capsaicin has the property of dilating blood vessels. However, this compound is highly irritating for the epidermis. It is therefore impossible to use it without giving rise to disorders affecting the epidermis.

Mustard essence and ginger essence are also known as blood-stream promoters, but the expected effect is neither satisfactory, nor durable.

JP Patent application 2001-316317 describes a blood-stream promoters which is a methoxy-phenolic ether of formula (I): 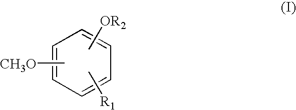
wherein:
   R₁ is a hydrogen or a methoxy group;
   R₂ is a C₁-C₁₈ alkyl group or a C₇-C₂₄ arylalkyl group.

A specific family of compounds of formula (I) consists of the dimethoxylated compounds of formula (I) (R₁=methoxy), in which R₂ is an alkyl group having at least 4 carbon atoms. In particular, in the described 3,5-dimethoxylated compounds, R₂ is a C₄, C₆-C₁₅, C₁₇ or C₁₈ alkyl group.

This patent application does not describe dimethoxylated compounds of formula (I) in which R₂ is an alkyl group containing 1 to 3 carbon atoms.

### SUMMARY OF THE INVENTION

Quite surprisingly, the present inventors now found that the compound of formula (A) hereunder shows a blood-stream promoting effect without irritating the epidermis.

Thus, the present invention has as its objects
(1) a method for topically promoting blood stream of a subject, which comprises applying at least one compound of formula (A): 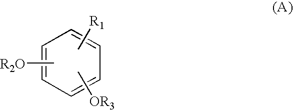
wherein
R₁ represents a hydrogen, a hydroxy group, a C₁-C₄ alkyl group or a C₁-C₃ alkoxy group, and
R₂ and R₃, each independently represent a hydrogen or a C₁-C₄ alkyl group, to the subject;
(2) the method according to 1 above, wherein R₂ and R₃, each represents a methyl group;
(3) the method according to 2 above, wherein R₁ represents a C₁-C₄ alkyl group;
(4) the method according to 2 above, wherein R₁ represents a C₁-C₃ alkoxy group;
(5) the method according to 2 above, wherein said compound of formula (A) is 1,3-dimethoxy-5-methylbenzene, 1,3,5-trimethoxybenzene or a mixture thereof;
(6) the method according to 1 above, wherein R₁ represents a hydroxy group, and R₂ and R₃, each represent a hydrogen;
(7) the method according to 6 above, wherein said compound of formula (A) is 1,3,5-trihydroxybenzene;
(8) the method according to 1 above, wherein said compound of formula (A) is 1,3-dimethoxy-5-methylbenzene, 1,3,5-trimethoxybenzene, 1,3,5-trihydroxybenzene, or a mixture thereof;
(9) the method according to 1 above, said compound of formula (A) being applied to the subject in the form of a topical composition;
(10) the method according to 9 above, wherein said composition comprises said compound of formula (A) in an amount of from 0.001% to 1% by weight based on the total weight of the composition;
(11) the method according to 9 above, wherein said composition is applied to skin; and
(12) the method according to 11 above, wherein the skin is scalp.

### DETAILED DESCRIPTION OF THE INVENTION

Particularly preferred compounds according to the present invention are compounds of formula (A) in which R₁ represents a C₁-C₄ alkyl group, preferably a methyl group or a C₁-C₃ alkoxy group, preferably a methoxy group, and R₂ and R₃, each represent a methyl group; or mixtures thereof.

Among these compounds, 1,3,5-trimethoxybenzene (hereinafter referred to as TMB), 1,3-dimethoxy-5-methylbenzene or mixtures thereof are particularly preferred.

Other compounds preferred with a view to carrying out the present invention are the compounds of formula (A) in which R₁ represents a hydroxy group, R₂ and R₃, each represent a hydrogen; or mixtures thereof.

Among these compounds, 1,3,5-trihydroxybenzene is particularly preferred.

Advantageously, there may be used a mixture of one or more methoxylated compounds of formula (A) with one or more hydroxylated compounds of formula (A). Particularly, mixtures of 1,3,5-trimethoxybenzene and/or 1,3-dimethoxy-5-methylbenzene with 1,3,5-trihydroxybenzene are preferable.

In the present description, “promoting blood stream” means promoting the blood stream, especially at a topical area, of the subject by applying at least one compound of formula (A) to the subject, and specifically, the blood-stream promoting effect according to the invention can be observed by the method described in the undermentioned test.

In the present invention, subjects of which the blood stream is promoted are animals, preferably mammals, and more preferably human.

According to the present invention, “topical compositions” are the cosmetic compositions or pharmaceutical compositions as defined below.

The topical composition according to the invention comprises at least one compound of formula (A) and the other ingredient. Specifically, as the example of the topical composition, there can be mentioned a topical composition which comprises the compound of formula (A) and a carrier or diluent which is acceptable for the topical composition.

As the examples of the carrier or diluent which is acceptable for the topical composition, solvents which can dissolve the compound of formula (A), such as ethanol, isopropanol, ethylene glycol, propylene glycol, butylene glycol, pentylene glycol, hexylene glycol, dipropylene glycol and the like, can be mentioned.

“Cosmetic compositions” in the present invention include cosmetic compositions, such as creams, lotions, shampoos, after-shampoos, as well as dermatological compositions for treatment of the skin, especially for the treatment of scalp.

In addition, the cosmetic composition according to the present invention may contain other ingredients such as excipients, additives, base, carrier or diluent. As the example of the additives for the cosmetic compositions, auxiliary agents such as a solubilizing agent, a moistening agent, a suspending agent, or an emulsifier; aromatics and preservatives can be mentioned.

“Pharmaceutical compositions” in the present invention include emulsions, solutions, suspensions, creams, ointments, compositions for plasters and the like.

In addition, the pharmaceutical composition according to the present invention may contain other ingredients such as excipients, additives, base, carrier or diluent. As the example of the additives for the pharmaceutical compositions, auxiliary agents such as a solubilizing agent, a moistening agent, a suspending agent, or an emulsifier; aromatics and preservatives can be mentioned.

The compounds of formula (A) are available from commercial sources.

They can be readily manufactured by the methods described or referred to by Wei et al., Organic Preparations and Procedures International, Vol. 35 (2003), 225, herein incorporated by reference.

In order to obtain the blood-stream promoting effect, the compound of formula (A) to be used in the topical compositions is preferably in an amount of from 0.001% to 1% by weight based on the total weight of the composition.

The invention will now be described in more detail by the following non-limiting test.

Seventeen women aged from 20 to 30 years old applied a cream to the whole of the index finger of their left-hand, and the blood flow of a part of the median phalanx of the index finger of their left-hand was measured by Doppler (Omega Flow FLO-N1; Omega ware Inc.).

The cream used in these tests was a face cream containing the following ingredients:

| | | | % by |
|---|---|---|---|
| Ingredients | Chemical Composition | Marketed by | weight |
| PART A | | | |
| MONTANOV 202 | ARACHIDYL ALCOOL & | SEPPIC | 3.00 |
| | BEHENYL ALCOOL & | | |
| | ARACHIDYLGLUCOSIDE | | |
| LANOL P | GLYCOL PALMITATE | SEPPIC | 5.00 |
| SIMULSOL 165 | POLYETHYLENE GLYCOL | SEPPIC | 2.00 |
| | STEARATE 100 & | | |
| | GLYCERYL STEARATE | | |
| DUB VCI 10 | ISODECYL | STEARINERIE | 5.00 |
| | NEOPENTANOATE | DUBOIS | |
| LANOL 99 | ISONONYL | SEPPIC | 9.00 |
| | ISONONAOATE | | |
| PART B | | | |
| MICROPEARL M305 | CROSS-LINKED | SEPPIC | 2.00 |
| | POLYMETHYLMETHACRYLATE | | |
| SEPITONIC M3 | MAGNESIUM | SEPPIC | 1.00 |
| | ASPARTATE & ZINC | | |
| | GLUCONATE & COPPER | | |
| | GLUCONATE | | |
| SEPICALM S | SODIUM SARCOSINATE | SEPPIC | 2.00 |
| | & POTASSIUM | | |
| | ASPARTATE & | | |
| | MAGNESIUM ASPARTATE | | |
| EDETA BD | SODIUM | BASF | 0.20 |
| | ETHYLENEDIAMINE | | |
| | TETRAACETATE | | |
| WATER | | | 54.80 |
| PART C | | | |
| DC345 | CYLOMETHICONE | LAMBERT RIVIERE | 8.00 |
| SIMULGEL EG | COPOLYMER OF SODIUM | SEPPIC | 2.50 |
| | ACRYLATE/ | | |
| | ACRYLOYLDIMETHYL | | |
| | TAURATE & | | |
| | ISOHEXADECANE & | | |
| | POLYSORBATE 80 | | |
| PART D | | | |
| ALCOHOL 96° C. | ETHANOL | | 4.00 |
| SEPICIDE HB | PHENOXYETHANOL/ | SEPPIC | 0.50 |
| | METHYLPARABEN/ | | |
| | ETHYLPARABEN/PROPYL | | |
| | PARABEN/ | | |
| | BUTYLPARABEN | | |

This cream was prepared by first of all mixing the ingredients of part A at 75° C.

The Micropearl M305 was dispersed using magnetic stirring in water at 70 to 75° C. Subsequently, the remainder of the ingredients of part B were mixed with the dispersion of Micropearl M305 and heated until a homogeneous mixture was obtained.

Part A and part B were then mixed and then, while stirring moderately, the silicone DC 345 and the SIMULGEL EG were added one after the other.

Thereafter, ethanol and the preservative SEPICIDE HB, were added whilst stirring at 45 to 50° C.

The cream obtained was then poured into pots. This face cream, not containing any perfume, was used for the control experiments (product C1).

The same face cream was also used containing either 1% by weight of rose perfume (product T1), or 0.1% by weight of TMB (product T2).

The rose perfume used to prepare product T1 had the following composition:

| Ingredients | CAS N° | % by weight |
|---|---|---|
| Benzyl alcohol | 100-51-6 | 2.5 |
| Citral | 5392-40-5 | 0.2 |
| Citronellol | 106-22-9 | 7.5 |
| Eugenol | 97-53-0 | 0.4 |
| Geraniol | 106-24-1 | 11 |
| Geranyl acetate | 105-87-3 | 0.3 |
| Cis-3-hexen-1-ol | 928-96-1 | 0.2 |
| Ionone alpha | 127-41-3 | 0.5 |
| Ionone beta | 14901-07-6 | 2.3 |
| Iso cyclo citral | 1335-66-6 | 0.3 |
| Nerol Pure | 106-25-2 | 2.0 |
| Phenylacetaldehyde | 122-78-1 | 1.2 |
| Phenylethyl acetate | 103-45-7 | 6.5 |
| Phenyl ethyl alcohol | 60-12-8 | 62.2 |
| Triethyl citrate | 77-93-0 | 2.85 |

For each cream, the blood circulation was measured before application of the cream, immediately after its application, 30 minutes after application and finally 60 minutes after application.

The cream samples were applied to women on a random basis.

The blood flow before application of the cream was set to 100%.

The results obtained are presented together in the following tables I to III; they show a steady profile of blood flow higher for product T2 with respect to product T1 and for product T2 with respect to control product C1.

No advantage with respect to product C1 appears for product T1.

The results were studied using the signed ranking test of Wilcoxon [“Introduction to Medical Statistics—OUP—M. Bland, ISBN 0192624288”]. This test confirmed a significant difference with a confidence level of 95% for high blood flow with product T2 with respect to product C1 and with respect to product T1, 60 minutes after application of the cream and also a significant difference for higher blood flow for product T2 just after application.

According to this test, for a confidence level of 95%, the absolute differences W have the following meanings:
W≦34, W≧119 [symbol] significant difference

34<W<119 [symbol] no significant difference

TABLE I
Blood flow 60 minutes after application of cream.
Comparison of C1 with respect to T2.

| Test | | | | | |
|---|---|---|---|---|---|
| after 60 | | | | absolute | |
| minutes | C1 | T2 | difference | difference | ranking |
| n° 1 | 127.94 | 171.60 | −43.66 | 43.66 | 12 |
| n° 2 | 117.21 | 59.09 | 58.13 | 58.13 | 9 |
| n° 3 | 121.72 | 28.38 | 93.34 | 93.34 | 6 |
| n° 4 | 684.37 | 163.04 | 521.33 | 521.33 | 1 |
| n° 5 | 62.76 | 97.41 | −34.65 | 34.65 | 13 |
| n° 6 | 2.39 | 13.23 | −10.84 | 10.84 | 16 |
| n° 7 | 44.37 | 107.37 | −62.99 | 62.99 | 8 |
| n° 8 | 33.25 | 102.46 | −69.21 | 69.21 | 7 |
| n° 9 | 52.94 | 76.25 | −23.30 | 23.30 | 15 |
| n° 10 | 56.02 | 100.76 | −44.74 | 44.74 | 11 |
| n° 11 | 67.88 | 222.57 | −154.69 | 154.69 | 4 |
| n° 12 | 50.38 | 101.08 | −50.70 | 50.70 | 10 |
| n° 13 | 98.60 | 126.50 | −27.90 | 27.90 | 14 |
| n° 14 | 184.64 | 177.87 | 6.76 | 6.76 | 17 |
| n° 15 | 36.19 | 264.91 | −228.71 | 228.71 | 2 |
| n° 16 | 41.80 | 166.96 | −125.16 | 125.16 | 5 |
| n° 17 | 115.38 | 309.09 | −193.72 | 193.72 | 3 |
| | | | | Overall ranking | 33 |
| | | | | W = | |

TABLE II
Blood flow 60 minutes after application of cream.
Comparison of T1 with respect to T2.

| Test | | | | | |
|---|---|---|---|---|---|
| after 60 | | | | absolute | |
| minutes | T1 | T2 | difference | difference | ranking |
| n° 1 | 113.52 | 171.60 | −58.08 | 58.08 | 10 |
| n° 2 | 90.00 | 59.09 | 30.92 | 30.92 | 13 |
| n° 3 | 19.19 | 28.38 | −9.20 | 9.20 | 15 |
| n° 4 | 161.27 | 163.04 | −1.76 | 1.76 | 17 |
| n° 5 | 35.59 | 97.41 | −61.83 | 61.83 | 9 |
| n° 6 | 105.64 | 13.23 | 92.41 | 92.41 | 4 |
| n° 7 | 181.76 | 107.37 | 74.39 | 74.39 | 7 |
| n° 8 | 67.43 | 102.46 | −35.04 | 35.04 | 11 |
| n° 9 | 57.40 | 76.25 | −18.85 | 18.85 | 14 |
| n° 10 | 28.91 | 100.76 | −71.86 | 71.86 | 8 |
| n° 11 | 70.36 | 222.57 | −152.21 | 152.21 | 3 |
| n° 12 | 193.48 | 101.08 | 92.40 | 92.40 | 5 |
| n° 13 | 121.42 | 126.50 | −5.08 | 5.08 | 16 |
| n° 14 | 94.65 | 177.87 | −83.23 | 83.23 | 6 |
| n° 15 | 44.99 | 264.91 | −219.92 | 219.92 | 2 |
| n° 16 | 133.81 | 166.96 | −33.15 | 33.15 | 12 |
| n° 17 | 70.43 | 309.09 | −238.66 | 238.66 | 1 |
| | | | | Overall ranking | 29 |
| | | | | W = | |

TABLE III
Blood flow immediately after application of cream (“after”) compared to
blood flow 60 minutes after application (“after 60 minutes”).

| | | after | | absolute | |
|---|---|---|---|---|---|
| | after | 60 min | difference | difference | ranking |
| n° 1 | 163.71 | 171.60 | −7.89 | 7.89 | 14 |
| n° 2 | 170.00 | 59.09 | 110.92 | 110.92 | 4 |
| n° 3 | 261.32 | 28.38 | 232.94 | 232.94 | 1 |
| n° 4 | 87.88 | 163.04 | −75.15 | 75.15 | 6 |
| n° 5 | 61.42 | 97.41 | −36.00 | 36.00 | 10 |
| n° 6 | 7.24 | 13.23 | −5.99 | 5.99 | 16 |
| n° 7 | 119.06 | 107.37 | 11.69 | 11.69 | 12 |
| n° 8 | 152.28 | 102.46 | 49.81 | 49.81 | 8 |
| n° 9 | 68.69 | 76.25 | −7.55 | 7.55 | 15 |
| n° 10 | 91.16 | 100.76 | −9.60 | 9.60 | 13 |
| n° 11 | 218.71 | 222.57 | −3.86 | 3.86 | 17 |
| n° 12 | 65.73 | 101.08 | −35.35 | 35.35 | 11 |
| n° 13 | 81.27 | 126.50 | −45.23 | 45.23 | 9 |
| n° 14 | 111.35 | 177.87 | −66.53 | 66.53 | 7 |
| n° 15 | 76.52 | 264.91 | −188.39 | 188.39 | 2 |
| n° 16 | 62.44 | 166.96 | −104.52 | 104.52 | 5 |
| n° 17 | 154.29 | 309.09 | −154.80 | 154.80 | 3 |
| | | | | Overall ranking | 25 |
| | | | | W = | |

This application is based on French patent application No. 04/08725 filed on Aug. 6, 2004, the entire contents thereof being hereby incorporated by reference.

What is claimed is:

1. A method for topically promoting blood stream of a subject, which comprises applying at least one compound of formula (A): 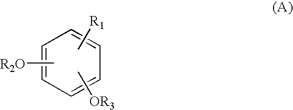
wherein
R₁ represents a hydrogen, a hydroxy group, a C₁-C₄ alkyl group or a C₁-C₃ alkoxy group, and
R₂ and R₃, each independently represent a hydrogen or a C₁-C₄ alkyl group, to the subject.

2. The method according to claim 1, wherein R₂ and R₃, each represent a methyl group.

3. The method according to claim 2, wherein R₁ represents a C₁-C₄ alkyl group.

4. The method according to claim 2, wherein R₁ represents a C₁-C₃ alkoxy group.

5. The method according to claim 2, wherein said compound of formula (A) is 1,3-dimethoxy-5-methylbenzene, 1,3,5-trimethoxybenzene or a mixture thereof.

6. The method according to claim 1, wherein R₁ represents a hydroxy group, and R₂ and R₃, each represent a hydrogen.

7. The method according to claim 6, wherein said compound of formula (A) is 1,3,5-trihydroxybenzene.

8. The method according to claim 1, wherein said compound of formula (A) is 1,3-dimethoxy-5-methylbenzene, 1,3,5-trimethoxybenzene, 1,3,5-trihydroxybenzene, or a mixture thereof.

9. The method according to claim 1, said compound of formula (A) being applied to the subject in the form of a topical composition.

10. The method according to claim 9, wherein said composition comprises said compound of formula (A) in an amount of from 0.001% to 1% by weight based on the total weight of the composition.

11. The method according to claim 9, wherein said composition is applied to skin.

12. The method according to claim 11, wherein the skin is scalp.
